(19) United States
(12) Patent Application Publication
(10) Pub. No.: US 2023/0233195 A1
(43) Pub. Date: Jul. 27, 2023

(54) NEEDLE APPARATUS, METHODS OF PERFORMING SAMPLING THEREWITH AND OF MANUFACTURING THEREOF

(71) Applicants: OULUN YLIOPISTO, Oulu (FI); AALTO-KORKEAKOULUSÄÄTIÖ SR, Aalto (FI)

(72) Inventors: Lukasz SURAZYNSKI, Oulu (FI); Miika NIEMINEN, Oulu (FI); Teemu MYLLYLÄ, Oulu (FI); Heikki J. NIEMINEN, Aalto (FI); Jouni RANTANEN, Aalto (FI); Ville HASSINEN, Oulu (FI); Markus MÄKINEN, Oulu (FI); Zuomin ZHAO, Oulu (FI)

(21) Appl. No.: 18/008,956

(22) PCT Filed: Jun. 7, 2021
(86) PCT No.: PCT/FI2021/050419

§ 371 Date: Dec. 7, 2022

(30) Foreign Application Priority Data
Jun. 8, 2020 (FI) 20205592

(51) Int. Cl. A61B 10/02

(52) U.S. Cl. CPC A61B 10/0275

(57) ABSTRACT

A needle apparatus (10) for taking a sample from inside of an object comprises a stylet (100), which comprises a longitudinal hollow (102). A transmission fiber arrangement (104) and a reception fiber arrangement (112) are on a bottom (126) of a surface (106) of the hollow (102). A first end (108) of the transmission fiber arrangement (104) is at an access tip (110) of the stylet (100). The transmission fiber arrangement (104) guides optical radiation from a second end (120) of the transmission fiber arrangement (104) to the first end (108) and output the optical radiation therefrom. A first end (114) of the reception fiber arrangement (112) is located at the access tip (110). The reception fiber arrangement (112) receives optical radiation from environment of the access tip (110) and guides the optical radiation therefrom to a second end (122) of the reception fiber arrangement (114) and outputs the optical radiation from there.

### FIELD

The invention relates to a needle apparatus, a method of performing sampling with the needle apparatus and a manufacturing method of the needle apparatus.

### BACKGROUND

Core needle biopsy is a method to investigate a tissue status of a patient. It is typically used in a procedure carried by interventional radiologists, where effects are observed by pathologists. It is a method in sampling many cancer suspects, therefore highly relevant for a cancer management. Nevertheless, biopsy plays a role afterwards a transplantation, and is performed, whenever there is even a slight suspect of a primary or secondary (metastases) of tumours.

As medical professionals have a limited insight in the tissue that is sampled, the sampling may fail or result in a poor sample. From various tissue conditions there are ones, which influence negatively a pathologist's decision. One factor is to have enough biological material. This is associated with a suitable size of the needle. Usually G18 (˜1.2 mm), G16 (˜1.6 mm) or G14 (˜2.1 mm) are utilized for procedure called Core Needle Biopsy (CNB). Unfortunately, even with a suitable amount of biological material histopathological investigation might be ruined by other factors, such as necrosis, too much healthy cells, a presence of fat, blood, non-representing connective tissue. Hence, an improvement for the biopsy process would be welcome.

### BRIEF DESCRIPTION

The present invention seeks to provide an improvement in the core needle biopsy.

The invention is defined by the independent claims. Embodiments are defined in the dependent claims.

### LIST OF DRAWINGS

Example embodiments of the present invention are described below, by way of example only, with reference to the accompanying drawings, in which

FIGS. 1A and 1B illustrate examples of a stylet and a cannula;

FIG. 1C illustrates an example of the stylet with optical fibers and electric wires inside a groove;

FIG. 1D illustrates an example of the stylet with optical fibers inside a groove;

FIG. 1E illustrates an example of the stylet with a hole within the stylet;

FIG. 1F illustrates an example of the stylet with electrodes;

FIG. 2 illustrates an example of a biopsy needle apparatus with an optical radiation source and a spectrometer;

FIG. 3 illustrates an example of a bundle of optical fibers which carry different band of optical radiation;

FIGS. 4A and 4B illustrate examples of curvatures of optical fibers of a transmission fiber arrangement and/or a reception fiber arrangement;

FIG. 5 illustrates an example of a data processing unit, which may reside in the spectrometer;

FIG. 6 illustrates an example of a biopsy procedure;

FIG. 7 illustrates an example of optical beams formed by fiber arrangements;

FIG. 8 illustrates an example of a flow chart of performing sampling from at least one organ of a patient with a biopsy needle apparatus; and

FIG. 9 illustrates of an example of a flow chart of a manufacturing method of a biopsy needle apparatus for taking a sample from at least one organ of a patient.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments to consist of only those features that have been mentioned and such embodiments may also contain features/structures that have not been specifically mentioned. All combinations of the embodiments are considered possible if their combination does not lead to structural or logical contradiction.

It should be noted that while Figures illustrate various embodiments, they are simplified diagrams that only show some structures and/or functional entities. The connections shown in the Figures may refer to logical or physical connections. It is apparent to a person skilled in the art that the described apparatus may also comprise other functions and structures than those described in Figures and text. It should be appreciated that details of some functions, structures, and the signalling used for measurement and/or controlling are irrelevant to the actual invention. Therefore, they need not be discussed in more detail here.

The following description is largely based on biopsy needles that are used to take a sample from tissue. However, the needle apparatus can be used to take samples from a variety of objects such as food, plants and any mainly solid material into which a needle can penetrate.

Core needle biopsy can be used to obtain small cylinders of tissue from a suspected lesion for histopathological diagnosis. Sampling procedure is carried by an interventional radiologist, and the diagnosis is set by a pathologist. Biopsies are essential for the preoperative diagnosis of over 99% of all cancers, and in addition, core needle biopsies are used in the diagnosis and follow-up of many chronic diseases, such as chronic liver diseases, transplanted organs

Radiologists should have a suitable techniques to get insight to the human body that allow accurate detection of various lesions for sampling. Yet, the resolution of present techniques does not allow determination of the quality of the samples, and therefore a significant proportion of samples taken are either insufficient for diagnosis or contain a suboptimal amount of a tumour.

The amount of the tissue is correlated to the calibre of the needle but even the widest calibre does not guarantee adequacy of the sample. Tumour necrosis, blood contamination, non-lesion sampling, and even tumour tissue composition may result in samples that are inadequate for diagnosis.

What is explained below improves and simplifies a job of interventional radiologists and provide information on tissue inside of a human body.

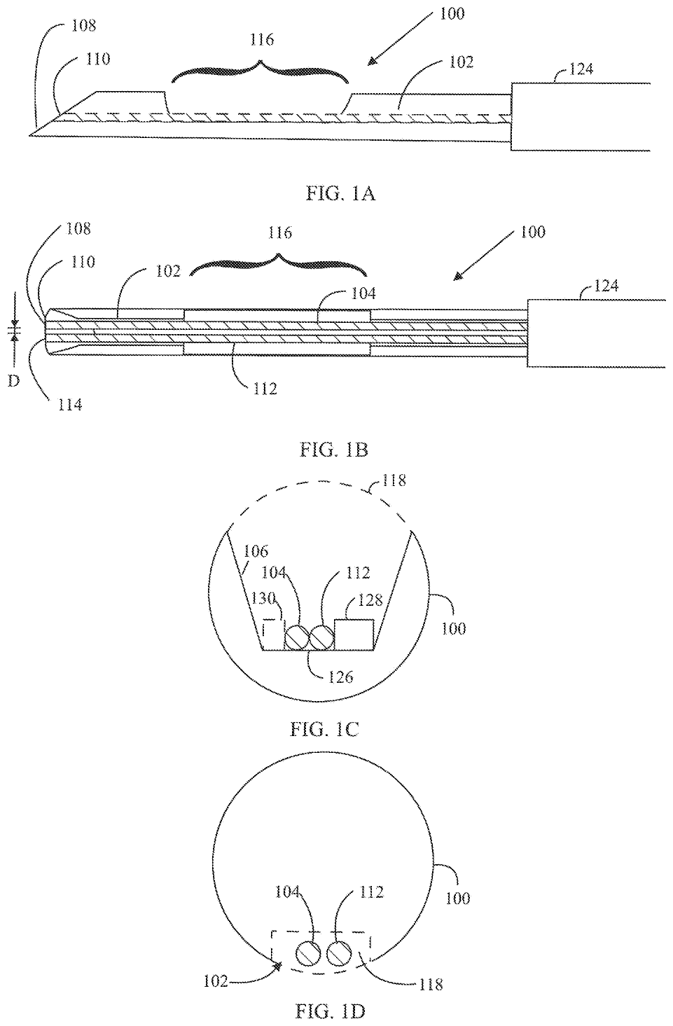

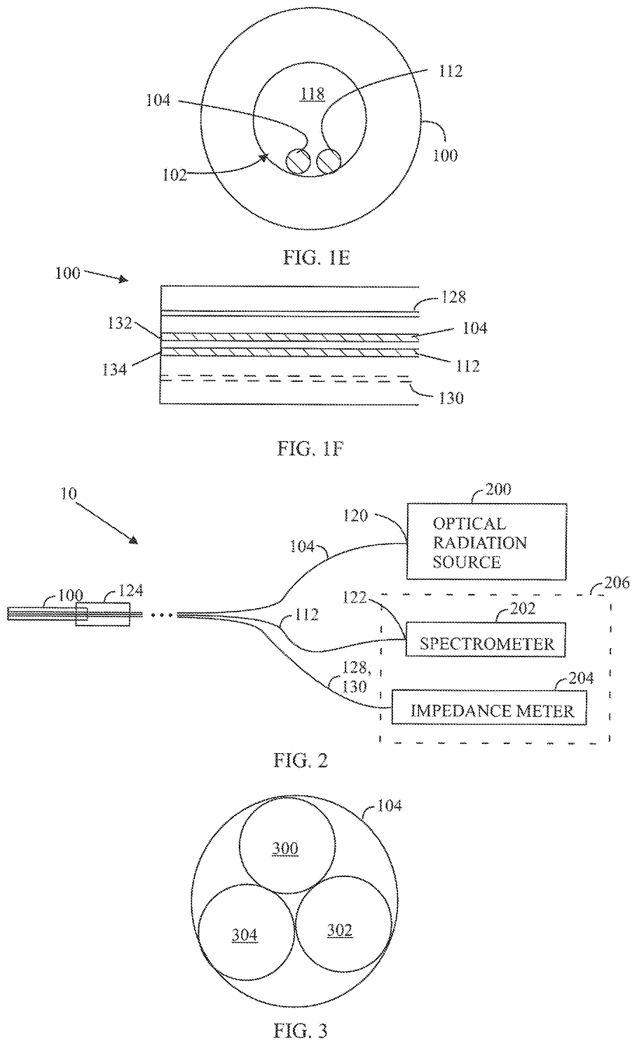

FIG. 1A, 1B, 1C, 1D, 1E and 1F illustrate an example of a stylet 100 of a biopsy needle apparatus 10 for taking a sample from at least one organ 602 of a patient 600 for laboratory testing. FIG. 1A illustrates an example of the stylet 100 from side. FIG. 1B illustrates an example of the stylet 100 from above. FIGS. 1C, 1D, 1E and 1F illustrate examples of a cross section of the stylet 100. The stylet 100 is a fine, longitudinally hollow needle for taking a small tissue sample from the patient 600. The stylet 10 may reside inside a cannula 124 or the like from which it is pushed forward when the sample is taken. The material of the stylet 100 may comprise a metal, ceramic, glass, polymer, or the like for example. The metal may be stainless steel or titan, for example. The stylet 10 may be made of different material from the cannula 124. The material of the stylet 10 may be softer than that of the cannula 124, for example. In that way, it can protect the fiber arrangements from breaking because of a mechanical shock when the stylet 100 hits the object.

The stylet 100 comprises a longitudinal hollow 102, i.e. the hollow 102 extends within a structure of the stylet 100 in a direction of a length of the stylet 100. The hollow 102 may comprise a groove (see FIGS. 1C and 1D) or a hole (see FIG. 1E) that extends in a longitudinal direction through the stylet 100. The hole is encircled with the material of the stylet 100 in a direction perpendicular to a longitudinal axis of the stylet except at a cavity 116. The groove is not fully encircled with the material of the stylet 100 but is partially open at one side. The open side of the groove may be straight and parallel with the longitudinal axis of the stylet 100, or it may have a non-zero angle or curve with respect to the longitudinal axis of the stylet 100. The hollow 102 comprises a channel for an optical measurement. The hollow 102 may also be a channel for one or more electric wires and their electrodes.

The biopsy needle apparatus 10 comprises at least one transmission fiber arrangement 104, which extends on a surface 106 of the longitudinal hollow 102 such that a first end 108 of the at least one transmission fiber arrangement 104 is located at an access tip 110 of the stylet 100, the at least one transmission fiber arrangement 104 guides optical radiation from a second end 120 of the at least one transmission fiber arrangement 104 to the first end 108 of the at least one transmission fiber arrangement 104 and outputs the optical radiation therefrom. When the stylet 100 is inside tissue of a patient 600 the optical radiation is directed to the tissue from the first end 108 of the at least one transmission fiber arrangement 104. The first end 108 of the at least one transmission fiber arrangement 104 may also be considered a proximal end.

The biopsy needle apparatus 10 comprises at least one reception fiber arrangement 112, which extends on the surface 106 of the longitudinal hollow 102 such that a first end 114 of the at least one reception fiber arrangement 112 is located at the access tip 110 of the stylet 100. A first end 114 of the at least one reception fiber arrangement 112 receives optical radiation from environment of the access tip 110 of the stylet 100, the at least one reception fiber arrangement 112 guides the optical radiation therefrom to a second end 122 of the at least one reception fiber arrangement 112, and outputs the optical radiation from there. When the stylet 100 is inside tissue of a patient 600 the optical radiation is received from the tissue. Namely, the tissue scatters the optical radiation directed thereto and a part of the scattered optical radiation is scattered backwards to the at least one reception fiber arrangement 112. The first end 114 of the at least one reception fiber arrangement 112 may, like the first end 108 of the at least one transmission fiber arrangement 104, be considered a proximal end. The second end 122 of the reception fiber arrangement 112 may be considered a distal end. In an embodiment, the transmission fiber arrangement 104 and/or the reception fiber arrangement 112 may be glued on the surface 106 of the hollow 102. In an embodiment, the transmission fiber arrangement 104 and/or the reception fiber arrangement 112 may be inside the filling material 118.

The hollow 102 may be etched or made using a laser machining, for example. In an embodiment, a cross-sectional shape of the hollow 102 may comprise a trapezoid. It enables easy placement of the at least one transmission fiber arrangement 104 and the at least one reception fiber arrangement 112 on the bottom 126 of the hollow 102. When the at least one transmission fiber arrangement 104 and the at least one reception fiber arrangement 112 are at the bottom of the hollow 102 that is a groove, the first end of the at least one transmission fiber arrangement 104 extends upto the access tip 110 of the stylet 100. In this manner, a large cavity 116 can be made which allows a larger sample from the tissue and thus a more accurate and reliable analysis of the sample. Immobility of the fiber arrangements 104, 112 may additionally increase the reliability.

In an embodiment, a cross-sectional shape of the hollow 102 may comprise triangle, where the at least one transmission fiber arrangement 104 and the at least one reception fiber arrangement 112 may be laid on the top of each other. This version ensure close distance between the at least one transmission fiber arrangement 104 and the at least one reception fiber arrangement 112, which are anyway placed on the bottom 126.

In an embodiment, a cross-sectional shape of the hollow 102 may comprise rounded corners. This is easy to manufacture, no special features rather than such dimensions are easier to achieve, when molding metal by pulling.

In an embodiment, a cross-sectional shape of the hollow 102 may comprise a circle. A circular cross-section allows additional space for sampling when compared with other shapes.

In general, different hollow shapes may be adopted in order to optimize needle stiffness reduction caused by placing the hollow 102 inside of the stylet 100.

In an embodiment, the biopsy needle apparatus 10 may comprise a cavity 116, which is a cut-off in a perpendicular direction to a longitudinal axis of the stylet 100, at a distance from the access tip 110. The cavity 116 may be used to take a sample from the at least one organ 602.

The at least one transmission fiber arrangement 104 and the at least one reception fiber arrangement 112 may be located in a predetermined manner with respect to each other. The predetermined manner of their location has an effect on an optical operation of the biopsy needle apparatus 10 when a sample is taken from the at least one organ 602.

In an embodiment, the hollow 102 with fiber arrangements 104, 112 and potentially the one or more electric wires 128, 130 may be filled with a filling material 118, which follows an outer contour of the stylet 100. The filling with the filling material 118 is thus matched with a height of the cavity 116 at the cavity 116 and a height of the hollow 102 through its full length. This means that a shape of the filling material 118 is matched with an outer contour of the stylet 100.

In an embodiment, the filling material 118 may improve stiffness of the stylet 100. Stiff enough a stylet 100 may be desirable in order the stylet 100 not to bend or break when it is stung into a patient 600. The filling material 118 may decouple the transmission fiber arrangement 104 and the reception fiber arrangement 112, which may be bridle, from a mechanical shock caused to the stylet 100 when it is shot to a patient 600. The filling material 118 may also allow embedding and positioning of the transmission fiber arrangement 104 and the reception fiber arrangement 112 (and electric wires 128, 130) into the hollow 102. A mechanical impedance of the filling material 118 may be higher or lower than a mechanical impedance of the transmission fiber arrangement 104, the reception fiber arrangement 112 and/or the stylet 100. A difference or a mismatch in the mechanical impedance decouples mechanical impacts from the fiber arrangements which may easily break. In this manner, these material may be mismatched mechanically which allows bending of the stylet 100 without causing stress to the transmission fiber arrangement 104 and the reception fiber arrangement 112. Namely, excess stress could cause mechanical failure of the transmission fiber arrangement 104 or the reception fiber arrangement 112, leading to failure to conduct the optical measurement correctly. In an embodiment, the filling material may be air, but it may instead also be solid material and/or liquid.

In an embodiment, the filling material 118 may comprise at least one polymer, rubber and/or silicone to prevent optical fiber fractures during sampling procedure and still strengthening the stylet 100.

In an embodiment, the at least one transmission fiber arrangement 104 comprises a single optical fiber, and the at least one reception fiber arrangement 112 comprises a single optical fiber. In an embodiment, the single fiber has a diameter of about 140 μm, for example.

In an embodiment, the at least one transmission fiber arrangement 104 may comprise a plurality of optical fibers. In a corresponding manner, the at least one reception fiber arrangement 112 may comprise a plurality of optical fibers. In this embodiment, the at least one transmission fiber arrangement 104 may comprise an optical fiber bundle, and the at least one reception fiber arrangement 112 may comprise an optical fiber bundle.

In an embodiment, the at least one transmission fiber arrangement 104 may transmit at least one of the following: ultraviolet light, visible light and infrared light. In an embodiment, the at least one transmission fiber arrangement 104 may transmit all the three different regions of light simultaneously. In an embodiment, the at least one transmission fiber arrangement 104 may transmit all the three different regions of light one after another.

FIG. 2 illustrates an example of the biopsy needle apparatus 10, where the second end 120 of the at least one transmission fiber arrangement 104 may be connected with a light source 200. The second end 120 of the at least one transmission fiber arrangement 104 may, like the second end 122 of the at least one reception fiber arrangement 112, be considered a distal end. The at least one transmission fiber arrangement 104 guides the light of the light source 200 to the access tip 110 of the stylet 100. The first end 114 of the reception fiber arrangement 112 may collect the scattered light from tissue and the reception fiber arrangement 112 may guide the light to the second end 122 of the reception fiber arrangement 112 and output the light to spectral analysis. The spectral analysis may be performed using a spectrometer 202.

In an embodiment, the light source 200 may output the three different regions of light: ultraviolet light, visible light and infrared light. In an embodiment, the light source 200 may comprise LEDs (light emitting diodes). In an embodiment, at least one of the LEDs may emit ultraviolet light, at least one LED may emit visible light and at least one LED may emit infrared light. In an embodiment, the light source may include at least one incandescent lamp, halogen lamp, electric discharge lamp, in addition to or instead of the LEDs, for example. In an embodiment, UV-light may be emitted from a germicidal lamp.

In an embodiment an example of which is illustrated in FIG. 2, the biopsy needle apparatus 10 may comprise an optical radiation source 200, which feeds the optical radiation to the at least one transmission fiber arrangement 104 through the second end 120 of the at least one transmission fiber arrangement 104. In an embodiment, the biopsy needle apparatus 10 may comprise a measuring arrangement 206.

In an embodiment, the measuring arrangement 206 of the biopsy needle apparatus 10 may comprise a spectrometer 202, which performs a spectral analysis of the optical radiation received from the second end 122 of the at least one reception fiber arrangement 112. In an embodiment, the spectrometer 202 may record spectral data for performing the spectral analysis later. In an embodiment, the spectrometer 202 may record spectral data for transferring the spectral data to a separate spectral analysis equipment in a wired or wireless manner.

In an embodiment, the measuring arrangement 206 of the biopsy needle apparatus 10 may comprise an impedance meter 204 which is connected with the at least one electric wire 128, 130 and potentially the stylet 100 or the filling material 118. The impedance meter 204 may perform an analysis of the impedance of the tissue during the biopsy operation. The impedance is an effective resistance of a direct or alternating electric current through the tissue between electrodes, which are formed of the at least one electric wire 128, 130 and potentially the stylet 100 or the filling material 118.

In an embodiment an example of which is illustrated in FIG. 3, the at least one transmission fiber arrangement 104 may comprise a plurality of optical fibers. At least one first optical fiber 300 of the optical fiber bundle of the at least one transmission fiber arrangement 104 may transmit ultraviolet light, at least one second optical fiber 302 of the at least one transmission fiber arrangement 104 may transmit visible light, and at least one third optical fiber 304 of the at least one transmission fiber arrangement 104 may transmit infrared light.

In an embodiment an example of which is illustrated in FIG. 1C, the at least one transmission fiber arrangement 104 and the at least one reception fiber arrangement 112 may be side by side in a physical contact with each other at a bottom 126 of the hollow 102.

In an embodiment an example of which is illustrated in FIG. 1B, the at least one transmission fiber arrangement 104 and the at least one reception fiber arrangement 112 may be spaced from each other in a predefined manner at a bottom 126 of the hollow 102. The spacing may be based on an optical operation of the biopsy needle apparatus 10 during a biopsy. The at least one transmission fiber arrangement 104 and the at least one reception fiber arrangement 112 may have a predefined distance D therebetween, for example. The distance D may be adaptable and/or adjustable.

In an embodiment an example of which is illustrated in FIG. 5, the measuring arrangement 206 may comprise one or more processors 500, and one or more memories 502 including a computer program code. The one or more memories 502 and the computer program code may be configured to, with the one or more processors 500, cause the spectrometer 202 at least to perform the spectral analysis of the optical radiation received from the second end 122 of the at least one reception fiber arrangement 112.

In an embodiment, the one or more memories 502 and the computer program code may be configured to, with the one or more processors 500, cause the impedance meter 204 at least to perform the impedance analysis of the electrical signals received from the two electric wire 128, 130 and/or at least one of the electric wires 128, 130 and the stylet 100, which is electrically conductive. The measuring arrangement 206 may thus perform both the spectral analysis and the impedance analysis instead of performing either the spectral analysis or the impedance analysis.

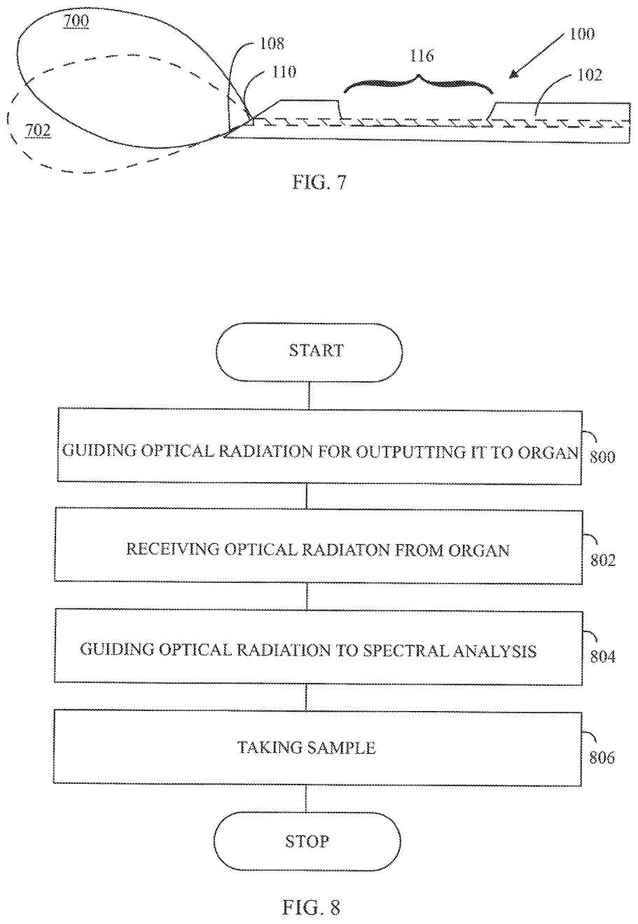

The data processing unit comprising the at least one processor 500 and the at least one memory may include an automated classification algorithm. It may be based on neural networks, which is a method of artificial intelligence (AI). A database having information obtained from a large number of biopsy measurements may be valuable for future collaborators as it might be utilized and scaled with other machine learning algorithms. The AI in this document may refer to or include also machine learning, neural network and/or deep learning. I analytics may perform the measurements in real time, and it may determine tissues and/or tissue types. It may also make measurements accurate. The AI analytics may be based on pattern recognition, for example. The AI analysis may be framed into figures (system diagram). In order to have a good utilization of the AI power, the biopsy needle apparatus may be connected with the Internet or some other data network, which has a database of the biopsy needle measurement or other measurements. Based on the data in the database, the needle apparatus may calibrate itself. The Al is illustrated in FIG. 6 within the user interface 504.

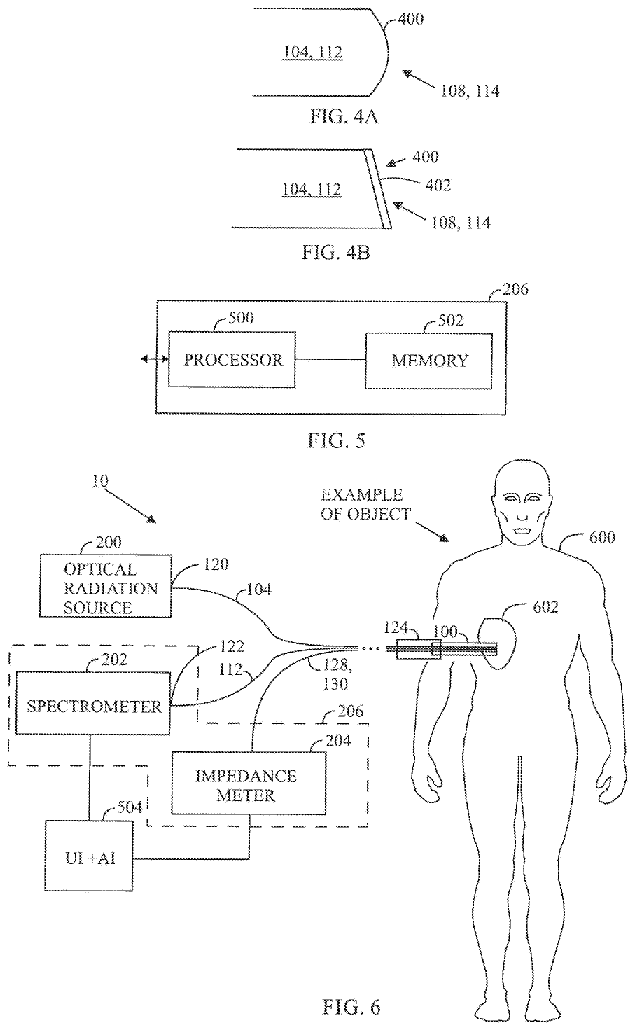

In an embodiment examples of which are shown in FIGS. 4A and 4B, a curvature of a polished surface 400 of the first end 108 of the transmission fiber arrangement 104 and a shape of the access tip 110 may have a predetermined dependence for directing the optical radiation to the tissue. That is, they may be matched together. In a corresponding manner, a curvature of a polished surface 400 of the first end 114 of the reception fiber arrangement 112 and a shape of the access tip 110 may have a predetermined dependence for directing the optical radiation to the tissue. In an embodiment corresponding to those of FIGS. 4A and 4B, a curvature of a polished surface of the first end 114 of the reception fiber arrangement 112 and the shape of the access tip 110 may be matched together for receiving the optical radiation from the tissue. When the access tip 110 of the stylet 100 is oblique, optical radiation cannot be as well directed to a longer sides of the access tip 110 as to a shorter side of the access tip 110. The corresponding applies to the reception of the optical radiation. Immobilized fiber arrangements 104, 112 in the groove 102 ensure a good direction of both a transmission beam and a reception beam of the optical radiation. The direction of the optical beams can further be secured, improved and made more precise by polishing the first ends 108, 114, which makes radiation curve within the tissue more suitable for the measurement. It may also cause a large illumination area of the tissue. This in turn allows to see and/or analyze where to take a sample. This in combination with the feature that the ends 108, 114 of fiber arrangements extend upto the access tip 110 of the stylet 100 allows an effective way of taking a sample by reducing cross talk, for example.

In an embodiment, the polished surface 400 of the at least one transmission fiber arrangement 104 and/or the at least one reception fiber arrangement 112 may be coated with a layer 402 (an example of the layer 402 is shown only in FIG. 4B although the layer 402 is generally applicable). The layer may act as an optical filter, which band passes a suitable band, and/or a distributor of light, which may direct, scatter and/or diffuse light, to or from the at least one organ 602. The layer may comprise metal and/or ink.

In an embodiment, the first end 108 of the at least one transmission fiber arrangement 104 and/or the first end 114 of the at least one reception fiber arrangement 112 may move within the hollow 102 for varying optical interconnection therebetween via tissue of the at least one organ 602. The movement of the at least one transmission fiber arrangement 104 may comprise rotation around a longitudinal axis of the at least one transmission fiber arrangement 104. The movement of the at least one reception fiber arrangement 112 may comprise rotation around a longitudinal axis of the at least one reception fiber arrangement 112. Rotation may be realized using a screw that rotates the at least one transmission fiber arrangement 104 and/or the at least one reception fiber arrangement 112, for example. The screw may be rotated manually or automatically under control of a data processing unit of the at least one processor 500 and the at least one memory 502 with the at least one computer program code.

The movement of the at least one transmission fiber arrangement 104 and/or the at least one reception fiber arrangement 112 may comprise a linear movement back and forth in a direction of a longitudinal axis of the at least one transmission fiber arrangement 104 and/or the at least one reception fiber arrangement 112. By moving the at least one transmission fiber arrangement 104 illumination of the tissue may be varied or changed.

The linear movement may be realized using an electric motor/stepper motor with stable and slow rotation for pulling the at least one transmission fiber arrangement 104 and/or the at least one reception fiber arrangement 112. In this manner a position of the fiber tips 108 and/or 114 are known. Alternatively or additionally, the at least one transmission fiber arrangement 104 and/or the at least one reception fiber arrangement 112 may be pulled manually.

The movement of the at least one transmission fiber arrangement 104 may comprise bending the at least one transmission fiber arrangement 104. When the at least one transmission fiber arrangement 104 is bent, the first end 108 of the transmission fiber arrangement 104 may move linearly sideways. The movement of the at least one reception fiber arrangement 112 may comprise bending the at least one reception fiber arrangement 112. When the at least one reception fiber arrangement 112 is bent, the first end 114 of the reception fiber arrangement 112 may move linearly sideways.

When the movement is combined with the curvature of a polished surface 400 of the first end 108 of the transmission fiber arrangement 104 and/or the curvature of a polished surface of the first end 114 of the reception fiber arrangement 112, a wider view of the tissue may be obtained.

In an embodiment, the transmission fiber arrangement 104 may output excitation light, and the reception fiber arrangement 112 may receive at least one of the following caused by the excitation light from the at least one organ 602 in conjunction with a biopsy sampling: Raman-light and fluorescent light. The Raman-light and/or the fluorescent light may be used to obtain important information on the at least organ 602.

In an embodiment an example of which is illustrated in FIG. 1D, the biopsy needle apparatus 10 comprises at least one electric wire 128, 130 on the surface 106 of the longitudinal hollow 102 such that a first end 132,134 of the at least one electric wire 128, 130 may be located at an access tip 110 of the stylet 100. In this example a cross-section of the groove 102 is smaller than that illustrated in FIG. 1C. The filling material 118 may keep the transmission fiber arrangement 104 and the reception fiber arrangement 112 in their places with a non-zero distance therebetween in this example.

In an embodiment an example of which is illustrated in FIG. 1E, the biopsy needle apparatus 10 comprises at least one electric wire 128, 130 on the surface 106 of the longitudinal groove 102 such that a first end 132,134 of the at least one electric wire 128, 130 may be located at an access tip 110 of the stylet 100. In an embodiment, the stylet 100 may be or comprise an opposite electrode to the at least one electric wire 128, 130, and a material of the opposite electrode is then electrically conductive. In an embodiment, the stylet 100 may be made of or comprise stainless steel, which is electrically conductive. Then the at least one electric wire 128, 130 is electrically insulted from the material of the opposite electrode of the stylet 100.

In an embodiment, the filling material 118 may be or comprise an opposite electrode to the at least one electric wire 128, 130, and a material of the opposite electrode is then electrically conductive. In this embodiment, the stylet 100 may be made of electrically insulating material.

In this manner, an electric measurement of the at least one organ 602 may be performed at the access tip 110 of the stylet 100. That may be called a bio-impedance measurement. Alternatively or additionally, the first end 132,134 of the at least one electric wire 128, 130 may be located inside of the cavity 116 (used for sampling) in order to enable a post-sampling measurement of a sample, which is in the cavity 116.

In an embodiment, the at least one electric wire 128, 130 may be used for cauterization in order to reduce or prevent bleeding or spreading liquid or cells within the body of the patient 600 after a sample has been taken. The cells that might spread may cancer cells, for example. Instead of the mere electric wire, the needle may comprise a cauterization electrode for stopping bleeding.

In an embodiment, the bio-impedance may be used to measure water content of the at least one organ 602. Different organs or tissues may have different water contents, which may be used to recognize if a sample is taken correctly.

In an embodiment, the transmission fiber arrangement 104 and the reception fiber arrangement 112 at located in the access tip 110 of the stylet 100, and their alignment or a location with respect to each other may be important and affects crucial parameters of the optical measurement such as crosstalk and an amount of light delivered to the tissue. However, the location of first tips 108, 114 may give a radiologist information on upcoming tissue, e.g. necrosis, fat, fibrosis or a vital tissue structure such as a large vessel.

In an embodiment, an ultra-rapid spectroscopy may be adopted in order to perform a screening of shooting the stylet 100 through tissue across its depth during an extraction i.e. a period during which the stylet 100 passes through tissue of the patient 600. A result of analyzed information measured optically and/or electrically during the period as a function of depth of the tissue may be presented to a user of the biopsy needle apparatus 10 and/or the patient 600 in real time or within seconds after shooting procedure.

FIG. 6 illustrates an example of sampling. The stylet 100 is inserted in the at least one organ 602 of the patient 600 and the access tip 110 cuts the tissue when the stylet 100 penetrates into the tissue. The optical radiation source 200 illuminates the at least one organ 602 because the transmission fiber arrangement 104 guides the light from the optical radiation source 200 to the at least one organ 602. The spectrometer 202 forms a spectrum of the light scattered and/or reflected from the at least one organ 602 because the reception fiber arrangement 112 guides the light from the at least one organ 602. A result of the spectral analysis may be presented using a user interface 504, which may comprise a screen, a touchscreen and/or sound source like a loudspeaker, for example. The user interface 504 may output sound and/or light signals for the user. The user interface 504 may also comprise a button and/or a keyboard, the button, the keyboard and/or the touchscreen being used to input data to the spectrometer 202. The data processing unit comprising the at least one processor 500 and the at least one memory 502 may control the biopsy process automatically or on the basis of the instructions input through the user interface 504.

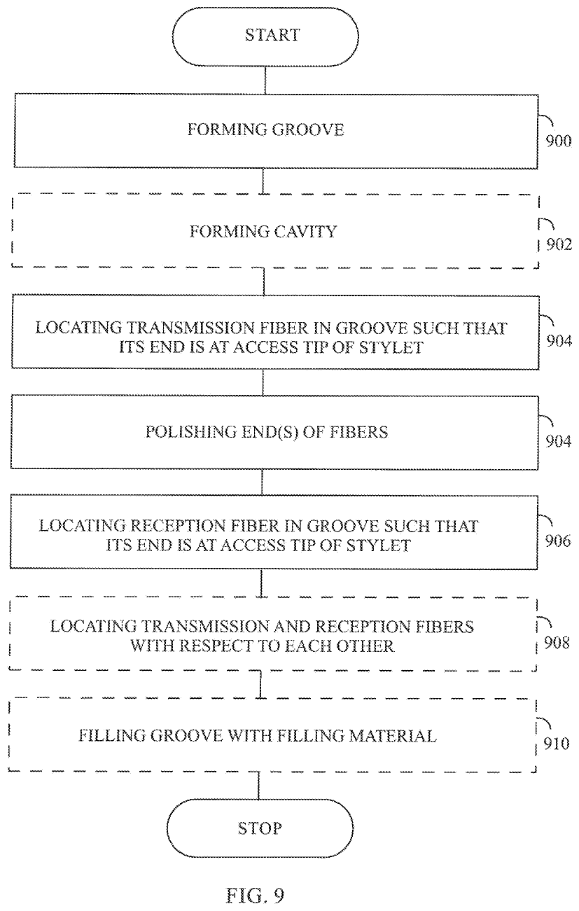

FIG. 7 illustrates an example of optical beams 700, 702 formed by fiber arrangements. The optical beam 702 illustrates a situation if the ends 108, 114 of the fiber arrangements were not polished to match with the access tip 110 of the stylet 100. The optical beam 702 would be directed straight ahead. By polishing the ends 108, 114 of the fiber arrangements the optical beam 700 is directed slightly upwards without significantly losing intensity directed ahead of the stylet 100. Because the optical beam 700, which represents both the transmission beam and the reception beam, is directed slightly to the side of the stylet 100 where the cavity 116 is, the optical beam illuminates the tissue or other section of the object in the area which the sample is taken from. This facilitates navigation within the object.

FIG. 8 illustrates on example of a method of performing sampling from at least one organ 602 of a patient 600 with a biopsy needle apparatus 10. In step 800, optical radiation is guided from a second end 120 of at least one transmission fiber arrangement 104 to a first end 108 of the at least one transmission fiber arrangement 104 for outputting the optical radiation therefrom to at least one organ 602, the at least one transmission fiber arrangement 104 extending on a surface 126 of a longitudinal groove 102 such that the first end 108 of the at least one transmission fiber arrangement 104 is located at an access tip 110 of the stylet 100.

In step 802, optical radiation is received from the at least one organ 602 with a first end 114 of at least one reception fiber arrangement 112, the first end 114 of the at least one reception fiber arrangement 112 locating at the access tip 110 of the stylet 100.

In step 804, the optical radiation is guided from the access tip 110 to a second end 122 of the at least one reception fiber arrangement 112 for outputting the optical radiation therefrom, the at least one reception fiber arrangement 112 extending on the surface 126 of the longitudinal groove 102, and the at least one transmission fiber arrangement 104 and the at least one reception fiber arrangement 112 being located in a predetermined manner with respect to each other in the groove 102. In an embodiment, the hollow may be filled with a filling material 118 following an outer contour of the stylet 100.

In step 806, a sample of the at least one organ 602 is taken with a cavity 116, which is a cut-off in a perpendicular direction to a longitudinal axis of the stylet 100, the cavity 116 locating at a distance from the access tip 110.

FIG. 9 illustrates an example of a manufacturing method of a biopsy needle apparatus 10 for taking a sample from at least one organ 602 of a patient 600. In step 900, a longitudinal hollow 102 is formed in a stylet 100 of the biopsy needle apparatus 10. In step 902, which is optional, a cavity 116 is formed in a perpendicular direction to a longitudinal axis of the stylet 100 at a distance from an access tip 110, the cavity 116 being configured to take a sample from the at least one organ 602.

In step 904, at least one transmission fiber arrangement 104 is located on a surface 126 of the longitudinal hollow 102 such that a first end 108 of the at least one transmission fiber arrangement 104 is located at the access tip 110 of the stylet 100, the at least one transmission fiber arrangement 104 being configured to guide optical radiation from a second end 120 of the at least one transmission fiber arrangement 104 to the first end 108 of the at least one transmission fiber arrangement 104 and output the optical radiation therefrom.

In step 906, at least one reception fiber arrangement 112 is located on the surface 126 of the longitudinal hollow 102 such that a first end 114 of the at least one reception fiber arrangement 112 is located at the access tip 110 of the stylet 100, the at least one reception fiber arrangement 112 being configured to receive optical radiation from environment of the access tip 110 of the stylet 100 and guide the optical radiation therefrom to a second end 122 of the at least one reception fiber arrangement 112 and output the optical radiation from there.

In step 908, the at least one transmission fiber arrangement 104 and the at least one reception fiber arrangement 112 may be located in a predetermined manner with respect to each other for an optical operation when a sample is taken from the at least one organ 602.

In step 910 which is an embodiment, the hollow 102 may be filled with a filling material 118 following an outer contour of the stylet 100. By using so-called smart core needle biopsy probe of the biopsy needle apparatus 10, it is possible to reduce a need for repeated patient admissions to core needle biopsy due to insufficient sampling. Automated algorithms working in real time may provide a feedback to a radiologist about the expected tissue type in area of sampling.

Reduction in the number of consequent biopsy procedures for the diagnosis will save money. It can be estimated that by using a smart core needle, about 15% reduction in the biopsy procedures could be achieved. This saves time from the admission to operation among cancer patients, which ultimately translates into improved survival. Furthermore, this technology is scalable to most of the parenchymal organs, such as liver, pancreas, breast, kidney, soft tissues and lymph nodes. The standalone spectrometric system may aid general core needle procedures, for example in liver. The optical light source 200 may provide broadband light, the spectrometer 202 and automated classification algorithm may differentiate tissue types even in real time.

Design of the biopsy needle apparatus 10 is compatible with most/all core needle biopsy arrangements, but it requires minor modification in their construction. It does not reduce their standard usability. The hollow such as the groove 102 may be laser machined rectangular or trapezoid-shaped into a stainless steel core needle biopsy stylet 100, for example. Regardless the manufacturing manner of the hollow 102 or its shape, the hollow 102 enables an introduction of the two or more optical channels. The hollow 102 is supplemented with biocompatible core which serves for certain purposes: strengthening the stylet 100 after removing some material, fixing a distance between the first tips 108, 114 of the at least one transmission fiber arrangement 104 and the at least one reception fiber arrangement 112 at the bottom 126 of the hollow 102, and enabling an automated manufacturing, especially during placement of optical fibers inside of the hollow. The at least one transmission fiber arrangement 104 and the at least one reception fiber arrangement 112 are used to illuminate tissue extracting spot and collect spectral data over this region, respectively. The spectral data may be used to detect necrosis, for example.

The biopsy needle apparatus 10 may be used as a single-use device in a corresponding manner to a conventional core needle biopsy probe. In order to achieve improvements in a biopsy procedure, a modification in core needle stylet is required. Such modification may be introduced in any general core needle biopsy as all of them include part responsible for cutting piece of the tissue—the stylet 100. It may be located inside of the cannula 124, movable-hollow part covering the stylet 100. Thus, the stylet 100 is compatible with both biopsy guns and manual biopsy syringes. Introduction of optical fibers into biopsy needle is known-to-art.

Nevertheless, the design of the stylet 100 and the other parts of the biopsy needle apparatus 10 are focusing on solving manufacturing issues and data gathering problems. Improvements are allowing tissue screening at the tip 110 of the stylet 100 and taking a biopsy sample based on the information received by the screening. Moreover, a mass production and a big reduction of costs may be possible.

The biopsy needle apparatus presented in this application is meant to be a universal system working with CNB devices and tools. The biopsy needle apparatus can be operated without additional training or teaching of the device itself and/or personnel, although the artificial intelligence of the biopsy needle apparatus may learn more during the use.

It should also be noted that a single fiber or fiber arrangement may be used for more than one measurement based on multiplexing. Different signals may be modulated differently for making their separation in reception possible. Possible modulations may be code division multiplexing or time division multiplexing, for example.

The A biopsy needle apparatus may be used to determine tissue characteristics such as antibodies, lipids, general blood quality, for example, in addition to mere determination of spectrum.

The application of the needle apparatus is not only in biopsy although the description is largely based on that. It can be utilized in surgical procedures. It is clear for a person skilled in the art that the needle apparatus can be applied to substances other than human or animal tissues. Fruits, jellies, soft bonbons and ice cream can also be measured, for example. That is, the needle apparatus is suitable for industry, too. In general, any material the needle can go inside can be measured.

Finally, the needle apparatus may visualize the area ahead of the tip of needle such that navigation within the object such as in the tissue is possible and easy. The Al may perform the visualization on a screen for the personnel taking the sample.

It will be obvious to a person skilled in the art that, as technology advances, the inventive concept can be implemented in various ways. The invention and its embodiments are not limited to the example embodiments described above but may vary within the scope of the claims.

1. A needle apparatus for taking a sample from inside of an object, wherein the needle apparatus comprises
a stylet , which comprises a longitudinal hollow,
at least one transmission fiber arrangement on a surface of a bottom of the longitudinal hollow such that a first end of the at least one transmission fiber arrangement is located at an access tip of the stylet, the at least one transmission fiber arrangement being configured to guide optical radiation from a second end of the at least one transmission fiber arrangement to the first end of the at least one transmission fiber arrangement and output the optical radiation therefrom,
at least one reception fiber arrangement on the surface of the bottom the longitudinal hollow such that a first end of the at least one reception fiber arrangement is located at the access tip (110) of the stylet (100), the at least one reception fiber arrangement being configured to receive optical radiation from environment of the access tip of the stylet and guide the optical radiation therefrom to a second end of the at least one reception fiber arrangement and output the optical radiation from there; and

2. The needle apparatus of claim 1, the hollow is filled with a filling material following an outer contour of the stylet, the filling with the filling material being matched with outer contour of the stylet, the filling material having a mechanical impedance different from a mechanical impedance of at least one of the following: the transmission fiber arrangement, the reception fiber arrangement and the stylet.

3. The needle apparatus of claim 1, wherein the needle apparatus comprises a cavity, which is a cut-off in a perpendicular direction to a longitudinal axis of the stylet, at a distance from the access tip, the cavity being configured to take a sample from the at least one organ.

4. The needle apparatus of claim 1, wherein the at least one transmission fiber arrangement comprises at least one optical fiber, and the at least one reception fiber arrangement comprises at least one optical fiber, and the at least one transmission fiber arrangement is configured to transmit at least one of the following: ultraviolet light, visible light and infrared light.

5. The needle apparatus of claim 4, wherein the at least one transmission fiber arrangement comprises a plurality of optical fibers, and at least one first optical fiber of the at least one transmission fiber arrangement is configured to transmit the ultraviolet light, at least one second optical fiber of the at least one transmission fiber arrangement is configured to transmit the visible light, and at least one third optical fiber of the at least one transmission fiber arrangement is configured to transmit the infrared light.

6. The needle apparatus of claim 1, wherein the at least one transmission fiber arrangement and the at least one reception fiber arrangement are side by side in a physical contact with each other at a bottom of the hollow. 7. The needle apparatus of claim 1, wherein a curvature of a polished surface of the first end of the transmission fiber arrangement and a shape of the access tip have a predetermined dependence for directing the optical radiation inside the object.

8. The needle apparatus of claim 1, wherein the transmission fiber arrangement is configured to output excitation light, and the reception fiber arrangement is configured to receive at least one of the following caused by the excitation light from the at least one organ in conjunction with a sampling: Raman-light and fluorescent light.

9. The needle apparatus of claim 1, wherein the needle apparatus comprises at least one electric wire on the surface of the longitudinal hollow such that a first end of the at least one electric wire is located at an access tip of the stylet.

10. The needle apparatus of claim 1, wherein the needle apparatus (100) comprises an optical radiation source, which is configured to feed the optical radiation to the at least one transmission fiber arrangement through the second end of the at least one transmission fiber arrangement and the needle apparatus comprises a spectrometer, which configured to perform a spectral analysis of the optical radiation received from the second end of the at least one reception fiber arrangement.

11. The needle apparatus of claim 10, wherein the spectrometer comprises one or more processors and one or more memories including a computer program code; and
the one or more memories and the computer program code being configured to, with the one or more processors, cause the spectrometer at least to perform the spectral analysis of the optical radiation received from the second end of the of the at least one reception fiber arrangement.

12. A method of performing sampling from an object with a needle apparatus, the method comprising
guiding optical radiation from a second end of at least one transmission fiber arrangement to a first end of the at least one transmission fiber arrangement for outputting the optical radiation therefrom to the at least one organ, the at least one transmission fiber arrangement extending on a surface of a bottom of a longitudinal hollow such that the first end of the at least one transmission fiber arrangement is located at an access tip of the stylet
receiving optical radiation from inside of the object with a first end of at least one reception fiber arrangement, the first end of the at least one reception fiber arrangement locating at the access tip of the stylet;
guiding the optical radiation from the access tip to a second end of the at least one reception fiber arrangement for outputting the optical radiation therefrom to spectral analysis, the at least one reception fiber arrangement extending on the surface of the bottom of the longitudinal hollow and the at least one transmission fiber arrangement and the at least one reception fiber arrangement being located in a predetermined manner with respect to each other in the hollow; and
taking a sample from inside of the object with a cavity which is a cut-off in a perpendicular direction to a longitudinal axis of the stylet, the cavity locating at a distance from the access tip.

13. A manufacturing method of a needle apparatus for taking a sample from an object, the method comprising forming a longitudinal hollow in a stylet of the needle apparatus;
locating at least one transmission fiber arrangement on a surface of the longitudinal hollow such that a first end of the at least one transmission fiber arrangement is located at the access tip of the stylet the at least one transmission fiber arrangement being configured to guide optical radiation from a second end of the at least one transmission fiber arrangement to the first end of the at least one transmission fiber arrangement and output the optical radiation therefrom;
locating at least one reception fiber arrangement on a bottom of the surface of the longitudinal hollow such that a first end of the at least one reception fiber arrangement is located at the access tip of the stylet, the at least one reception fiber arrangement being configured to receive optical radiation from environment of the access tip of the stylet and guide the optical radiation therefrom to a second end of the at least one reception fiber arrangement and output the optical radiation from there.
